(19) United States
(12) Patent Application Publication
(10) Pub. No.: US 2009/0175947 A1
(43) Pub. Date: Jul. 9, 2009

(54) PHARMACEUTICAL COMPOSITION FOR INJECTIONAL PARTICULARLY TARGETED LOCAL ADMINISTRATION

(76) Inventors: Ales Franc, Brno (CZ); Petr Sova, Hradec Kralove (CZ)

Correspondence Address: NOTARO & MICHALOS P.C., 100 DUTCH HILL ROAD, SUITE 110, ORANGEBURG, NY, 10962-2100, US

(21) Appl. No.: 12/305,308

(22) PCT Filed: Jun. 20, 2007
(86) PCT No.: PCT/CZ2007/000060

§ 371 Date: Mar. 12, 2009

(30) Foreign Application Priority Data
Jun. 20, 2006 (CZ) PV 2006-401

(51) Int. Cl. A61K 9/14; A61P 35/00

(52) U.S. Cl. 424/489

(57) ABSTRACT

The invention relates to a pharmaceutical composition for injectional, in particular targeted local administration, characterized in that it comprises a sterile suspension of platinum complex of general formula (I), wherein A and A′ independently of one another are an NH₃ group or an amino or diamino group containing 1 to 18 carbon atoms, B and B′ independently of one another are a halogen atom or a hydroxy group or are an —O—C(O)—R or an —O—C(O)—R′ group wherein R and R′ independently of one another are hydrogen atom, an alkyl, alkenyl, aryl, aralkyl, alkylamino or alkoxy group which groups contain 1 to 10 carbon atoms, or functional derivatives of these groups, X and X′ independently of one another are a halogen atom or a monocarboxylate group containing 1 to 20 carbon atoms, or X and X′ together form a dicarboxylate group containing 2 to 20 carbon atoms, in a pharmaceutically acceptable hydrophilic or hydrophobic injection liquid phase, 100% of particles of the platinum complex of general formula I being of size smaller than 250 μm.

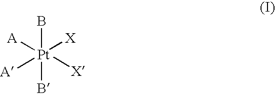

### FIELD OF THE INVENTION

The invention relates to a composition for injectional, particularly targeted local administration, enabling application of the active compound directly to the tumor site.

### BACKGROUND OF THE INVENTION

Platinum complexes are generally known as effective substances having broad spectrum of antitumor effect and they are thus utilized for treating a number of tumor diseases. So far, the therapeutic practice has used only complexes of bivalent platinum, especially cisplatinum, carboplatinum or oxaliplatinum. Bivalent platinum complexes are unstable in the gastrointestinal tract and/or are very poorly absorbed. This makes the use of bivalent platinum complexes in an oral, otherwise for the patient more acceptable dosage form, impossible. It has been found that some complexes of tetravalent platinum have not this disadvantage and retain their antitumor activity even when administered orally. These complexes of tetravalent platinum were disclosed as novel chemical compounds for oral administration in EP 0 328 274, EP 0 423 707 and PCT/CZ99/00015.

However, tetravalent platinum complexes exhibit very poor solubility in water (about 0.03 g/100 ml), low bulk density (about 0.2 g/ml), low tap density (about 0.4 g/ml), and a high electrostatic charge. The mentioned physical properties of the complexes represent an important problem in the preparation of their solid oral drug form. Moreover, complexes of tetravalent platinum are chemically unstable in contact with metals or with many currently used excipients. These problems are partially solved in PCT/CZ99/00015 which patent document describes the preparation of solid drug forms of specific tetravalent platinum complexes in the form of their inclusion complexes with cyclodextrins. According to the mentioned patent document, these inclusion complexes are obtained by reaction of cyclodextrins with tetravalent platinum complexes in an organic solvent and subsequent lyophilization, and are used for oral application. However, the employed amount of cyclodextrin markedly limits the content of the tetravalent platinum complex in the oral drug form, which represents a drawback. The obtained oral drug form thus has a relatively large volume and is difficult to swallow, making thus a single-dose oral application of greater amounts of tetravalent platinum complex impossible.

This disadvantage of oral forms of tetravalent platinum complexes could be eliminated by providing an injection form with sufficient content of tetravalent platinum complexes. From the state of the art it is evident that so far the preparation of such an injection form has not been satisfactorily solved. The aim of this invention is thus to provide a pharmaceutical composition for injectional administration that would contain sufficient, therapeutically effective, amount of tetravalent platinum complexes.

### SUMMARY OF THE INVENTION

Within the framework of the invention, it has been unexpectedly found that the below specified complexes of tetravalent platinum, contained in the pharmaceutical composition according to the invention, can be used not only in systemic, but also in local administration, which makes it possible to apply the tetravalent platinum complex directly at the tumor site. Because of cumulation of the tetravalent platinum complex at the tumor site, a lower therapeutic dose of the complex can be used in comparison with a systemically applied dose of the same therapeutic effect. Moreover, the tetravalent platinum complex is thus protected against a longer circulation in the bloodstream and against chemical and enzymatic cleavage occurring in contact with biological protecting systems present in biological liquids, particularly in digestive juices and in blood, or during passage through the liver. In addition, use of a pharmaceutical composition according to the invention makes it possible to achieve a protracted effect of the tetravalent platinum complex.

This invention thus relates to a pharmaceutical composition for injectional, in particular targeted local administration, characterized in that it comprises a sterile suspension of platinum complex of general formula I,

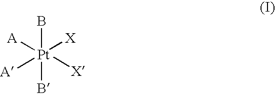

wherein
A and A′ independently of one another are an NH₃ group or an amino or diamino group containing 1 to 18 carbon atoms,
B and B′ independently of one another are a halogen atom or a hydroxy group or are an —O—C(O)—R or an —O—C(O)—R′ group wherein R and R′ independently of one another are hydrogen atom, an alkyl, alkenyl, aryl, aralkyl, alkylamino or alkoxy group which groups contain 1 to 10 carbon atoms, or functional derivatives of these groups,
X and X′ independently of one another are a halogen atom or a monocarboxylate group containing 1 to 20 carbon atoms, or X and X′ together form a dicarboxylate group containing 2 to 20 carbon atoms,
   in a pharmaceutically acceptable hydrophilic or hydrophobic injection liquid phase, 100% of particles of the platinum complex of general formula I being of size smaller than 250 μm.

Preferably, 90% of particles of the platinum complex of general formula I are of size smaller than 40 μm, preferably smaller than 10 μm.

Preferably, the weight ratio of the platinum complex of general formula I to the liquid phase is 1:100 to 30:100, more preferably 5:100 to 10:100.

Preferably, as a liquid phase the pharmaceutical composition contains a hydrophilic liquid phase selected from the group consisting of water, glycerol and propylene glycol.

Preferably, the liquid phase contains at least one stabilizer, in particular a peptizer and/or a viscosifier.

Preferably, as peptizer, the liquid phase contains a polyol compound, advantageously selected from the group consisting of lactose, fructose, mannitol and sorbitol.

Preferably, the weight ratio of the polyol compound to the platinum complex of general formula I is 0.1:1 to 10:1.

Preferably, as a viscosifier, the liquid phase contains a hydrophilic polymer, advantageously selected from the group consisting of polyvinylpyrrolidone, polyvinyl acetate, hydroxypropyl cellulose and hydroxypropyl methyl cellulose.

Preferably, the pharmaceutical composition contains 0.1 to 10% by weight of at least one viscosifier, based on the total weight of the composition.

Preferably, as the liquid phase, the pharmaceutical composition contains a hydrophilic liquid phase which is an aqueous buffer pH 4 to 8.

Preferably, as the liquid phase, the pharmaceutical composition contains a hydrophobic liquid phase which is an oleophilic medium, selected in particular from the group consisting of oil for injections, olive oil and sunflower oil.

The invention also relates to the above mentioned pharmaceutical composition as a drug for the treatment of malignant tumors.

In the following part, the invention will be explained in more detail using specific examples of execution which are of illustrative nature only and do not limit in any way the scope of the invention that is unequivocally defined by the appending Claims.

In these Examples, as a specific representative of platinum complex of general formula I serves the (OC-6-43)-bis(acetato)-(1-adamantylamine)-ammine-dichloroplatinum(IV) complex of code name LA-12 and of structural formula II:

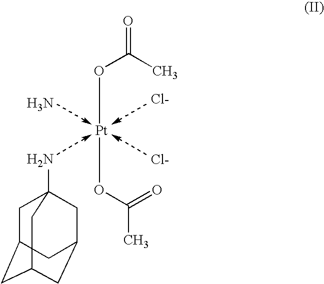

In the Examples, this specific platinum complex is denoted by its code name.

### EXAMPLES

### Example 1

### Preparation of Hydrophilic Suspension Injection Form of Platinum Complex LA-12

Mannitol (5 parts by weight) and hydroxypropyl methyl cellulose (1 part by weight) are dissolved in an aqueous buffer pH 4 (85 parts by weight) and the solution is sterilized by autoclaving. In the thus-obtained sterilized solution, platinum complex LA-12 (100% of particles smaller than 250 μm) is aseptically suspended. The resulting suspension is aseptically filled into sterile vials.

### Example 2

### Preparation of Hydrophobic Injection Form of Platinum Complex LA-12

Platinum complex LA-12, 100% particles of which are of size smaller than 250 μm (5 parts by weight), is aseptically suspended in oil for injections (95 parts by weight). The resulting suspension is then aseptically filled into sterile vials.

1. A pharmaceutical composition for injectional, in particular targeted local administration, characterized in that it comprises a sterile suspension of (OC-6-43)-bis(acetato)-(1-adamantylamine)-ammine-dichloroplatinum(IV) complex of structural formula II: 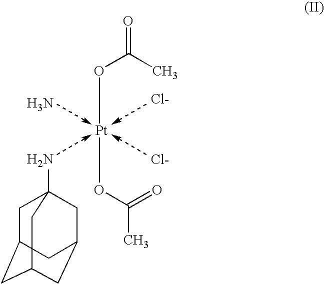
in a pharmaceutically acceptable hydrophilic or hydrophobic injection liquid phase, 100% of particles of the complex of structural formula II being of size smaller than 250 μm.

2. The pharmaceutical composition according to claim 1, characterized in that 90% of particles of the complex of structural formula II are of size smaller than 40 μm, preferable smaller than 10 μm.

3. The pharmaceutical composition according to claim 1, characterized in that the weight ratio of the complex of structural formula II to the liquid phase is 1:100 to 30:100.

4. The pharmaceutical composition according to claim 3, characterized in that the weight ratio of the complex of structural formula II to the liquid phase is 5:100 to 10:100.

5. The pharmaceutical composition according to claim 1, characterized in that as liquid phase it comprises a hydrophilic liquid phase selected from the group comprising water, glycerol and propylene glycol.

6. The pharmaceutical composition according to claim 1, characterized in that the liquid phase comprises at least one stabilizer, preferably peptizer and/or viscosifer.

7. The pharmaceutical composition according to claim 6, characterized in that as peptizer the liquid phase contains a polyol compound, preferably selected from the group comprising lactose, fructose, mannitol and sorbitol.

8. The pharmaceutical composition according to claim 7, characterized in that the weight ratio of the polyol compound to the complex of structural formula II is 0.1:1 to 10:1.

9. The pharmaceutical composition according to claim 6, characterized in that as viscosifier the liquid phase contains a hydrophilic polymer, preferably selected from the group comprising polyvinylprrolidone, polyvinyl acetate, hydroxypropyl cellulose and hydroxypropyl methyl cellulose.

10. The pharmaceutical composition according to claim 9, characterized in that it contains 0.1 to 10% by weight of at least one viscosifier, based on the total weight of the composition.

11. the pharmaceutical composition according to claim 1, characterized in that as a liquid phase it contains a hydrophilic liquid phase consisting of an aqueous buffer, pH4 to 8.

12. The pharmaceutical composition according to claim 1, characterized in that as a liquid phase it contains a hydophobic liquid phase consisting of an oleophilic medium, preferably selected from the group consisting of oil for injections, olive oil and sunflower oil.

13. A pharmaceutical composition according to claim 1, as a drug for the treatment of malignant tumors.
